(19) United States
(12) Patent Application Publication
(10) Pub. No.: US 2018/0280630 A1
(43) Pub. Date: Oct. 4, 2018

(54) APPARATUS AND METHOD FOR REMOVING AIR FROM A FLUID

(71) Applicant: Vanderbilt University, Nashville, TN (US)

(72) Inventors: Yandong Jiang, Brentwood, TN (US); Byron F. Smith, Nashville, TN (US); Lars Axel Strombergsson, Franklin, TN (US); Haoxiang Luo, Franklin, TN (US)

(21) Appl. No.: 15/943,913

(22) Filed: Apr. 3, 2018

Related U.S. Application Data
(60) Provisional application No. 62/481,350, filed on Apr. 4, 2017.

(51) Int. Cl. A61M 5/36; A61M 5/168; A61M 39/22; A61M 5/38

(52) U.S. Cl. CPC A61M 5/36; A61M 5/16809; A61M 39/22; A61M 2206/16; A61M 5/385; A61M 2039/226; A61M 5/365

(57) ABSTRACT

An apparatus for removing air from a fluid comprises a vertically oriented chamber having top and bottom chamber portions separated by a chamber sidewall at least partially defining a chamber interior having a chamber centerline. A fluid inlet provided through the chamber sidewall selectively permits ingress of fluid to the chamber interior. Fluid entering the chamber interior through the fluid inlet is directed laterally between the chamber sidewall and the chamber centerline. A fluid outlet located proximate the bottom chamber portion selectively permits egress of fluid from the chamber interior. An air release structure located proximate the top chamber portion selectively releases accumulated air from the chamber interior. Fluid entering the chamber interior through the fluid inlet achieves a vortex motion during travel downward to the fluid outlet. The vortex motion prompts release of air from the fluid, the air collecting adjacent the air release structure for selective release.

### RELATED APPLICATION

This application claims priority from U.S. Provisional Application No. 62/481,350, filed 4 Apr. 2017, the subject matter of which is incorporated herein by reference in its entirety.

### TECHNICAL FIELD

This disclosure relates to an apparatus and method for removing air from a fluid and, more specifically to a method and apparatus for automatically detecting and removing air from an IV line without removing liquid and with minimal or no interruption of forward flow.

### BACKGROUND

Air embolism is a preventable hospital-acquired condition that can result in serious harm, including death. However, there is no trend indicating the harm is decreasing within last 20 years. The causes of air embolism are numerous. Pressurized infusion or transfusion of either IV fluid or blood products seems the major cause of clinically significant air embolism. Air entering the blood of a patient tends to come from the dissolved air in the IV fluid or blood product which is de-aired during warming up toward the body temperature, existing air in the bag, and/or air that the provider unintentionally introduced into the bag or vascular access line. During pressurized infusion, air dissolved in the liquid or existing in the bag can be rapidly infused into the patient's blood stream and cause air embolism. In the last two decades, industry has developed multiple devices to overcome the problem.

In North America, the Belmont pump, available from Belmont Instrument Corporation of Billerica, Mass., is the most commonly used. The Belmont Pump can provide maximal infusion speed up to 750 ml/min, with temperature of the transfused liquid leaving the device at 38° C. and this device is equipped with automatic air detecting and removing system. It can automatically detect and remove air of 100 μL or greater. There is a new device, ClearLine MD, available from ClearLine MD of Woburn, Mass., with similar features to the Belmont pump. The manufacturer of the ClearLine MD device claims that it can provide a maximal speed of infusion up to 1000 ml/min and detect and remove air bubble as small as 50 μL. The issue remains in both these devices, though, that forward flow has to be terminated when the air bubble(s) is detected and removed. The devices are bulky and both the devices themselves and the associated disposables are expensive.

### SUMMARY

In an aspect, an apparatus for removing air from a fluid is provided. The apparatus comprises a vertically oriented chamber having longitudinally spaced top and bottom chamber portions separated by a chamber sidewall. The chamber sidewall at least partially defines a chamber interior having a longitudinally oriented chamber centerline. A fluid inlet is provided through the chamber sidewall and is configured to selectively permit ingress of fluid to the chamber interior. The fluid inlet is oriented at a tangent to the chamber sidewall, such that fluid entering the chamber interior through the fluid inlet is directed laterally between the chamber sidewall and the chamber centerline. A fluid outlet is located proximate the bottom chamber portion and is configured to selectively permit egress of fluid from the chamber interior. An air release structure is located proximate the top chamber portion and is configured to selectively release accumulated air from the chamber interior. Fluid entering the chamber interior through the fluid inlet achieves a vortex motion during travel downward to the fluid outlet. The vortex motion prompts release of air from the fluid. The accumulated air collects adjacent the air release structure for selective release.

In an aspect, a method of removing air from a fluid is provided. The method comprises providing an apparatus including a vertically oriented chamber with a chamber interior at least partially defined by longitudinally spaced top and bottom chamber portions separated by a chamber sidewall. The chamber sidewall at least partially defines the chamber interior. A fluid inlet is provided through the chamber sidewall. The fluid inlet is oriented at a tangent to the chamber sidewall. A fluid outlet is located proximate the bottom chamber portion. An air release structure is located proximate the top chamber portion. Ingress of fluid is selectively permitted to the chamber interior via the fluid inlet. Fluid entering the chamber interior through the fluid inlet is directed in a direction laterally between the chamber sidewall and a longitudinally oriented chamber centerline. Egress of fluid is selectively permitted from the chamber interior via the fluid outlet. A vortex motion is imparted to fluid traveling through the chamber interior between the fluid inlet and the fluid outlet. With the vortex motion, release of air is prompted from the fluid traveling through the chamber interior to produce accumulated air. The accumulated air is collected in the chamber interior adjacent the air release structure. The accumulated air is selectively released from the chamber interior via the air release structure.

### BRIEF DESCRIPTION OF THE DRAWINGS

For a better understanding, reference may be made to the accompanying drawings, in which:

FIG. 1 is a schematic side view of an aspect of the present invention in a first configuration;

FIG. 2 is a schematic side view of an aspect of the present invention in a second configuration;

FIG. 3 is a schematic side view of an aspect of the present invention in a third configuration;

FIG. 4 is a schematic side view of the aspect of FIG. 3;

FIG. 5 is a partial exploded perspective view taken from FIG. 4;

FIG. 6 is a schematic top view of an example fluid flow of any aspect of the invention;

FIG. 7 is a schematic side view of an example fluid flow of any aspect of the invention;

FIG. 8 is an example schematic diagram of any aspect of the invention;

FIG. 9 is an example schematic electrical diagram of any aspect of the invention; and

FIG. 10 is a schematic representation of an example fluid flow within a component of any aspect of the invention.

Description of Aspects of the Disclosure

Unless defined otherwise, all technical and scientific terms used herein have the same meaning as is commonly understood by one of skill in the art to which the present disclosure pertains.

As used herein, the term “subject” can be used interchangeably with the term “patient” and refer to any warm-blooded organism including, but not limited to, human beings, pigs, rats, mice, dogs, goats, sheep, horses, monkeys, apes, rabbits, cattle, farm animals, livestock, etc.

As used herein, the singular forms “a,” “an” and “the” can include the plural forms as well, unless the context clearly indicates otherwise. It will be further understood that the terms “comprises” and/or “comprising,” as used herein, can specify the presence of stated features, steps, operations, elements, and/or components, but do not preclude the presence or addition of one or more other features, steps, operations, elements, components, and/or groups thereof.

As used herein, the term “and/or” can include any and all combinations of one or more of the associated listed items.

As used herein, phrases such as “between about X and Y” can mean “between about X and about Y.”

It will be understood that when an element is referred to as being “on,” “attached” to, “connected” to, “coupled” with, “contacting,” etc., another element, it can be directly on, attached to, connected to, coupled with or contacting the other element or intervening elements may also be present. In contrast, when an element is referred to as being, for example, “directly on,” “directly attached” to, “directly connected” to, “directly coupled” with or “directly contacting” another element, there are no intervening elements present. It will also be appreciated by those of skill in the art that references to a structure or feature that is disposed “directly adjacent” another feature may have portions that overlap or underlie the adjacent feature, whereas a structure or feature that is disposed “adjacent” another feature might not have portions that overlap or underlie the adjacent feature.

Spatially relative terms, such as “under,” “below,” “lower,” “over,” “upper” and the like, may be used herein for ease of description to describe one element or feature's relationship to another element(s) or feature(s) as illustrated in the figures. It will be understood that the spatially relative terms can encompass different orientations of a device in use or operation, in addition to the orientation depicted in the figures. For example, if a device in the figures is inverted, elements described as “under” or “beneath” other elements or features would then be oriented “over” the other elements or features.

It will be understood that, although the terms “first,” “second,” etc. may be used herein to describe various elements, these elements should not be limited by these terms. These terms are only used to distinguish one element from another. Thus, a “first” element discussed below could also be termed a “second” element without departing from the teachings of the present disclosure. The sequence of operations (or steps) is not limited to the order presented in the claims or figures unless specifically indicated otherwise.

The invention comprises, consists of, or consists essentially of the following features, in any combination.

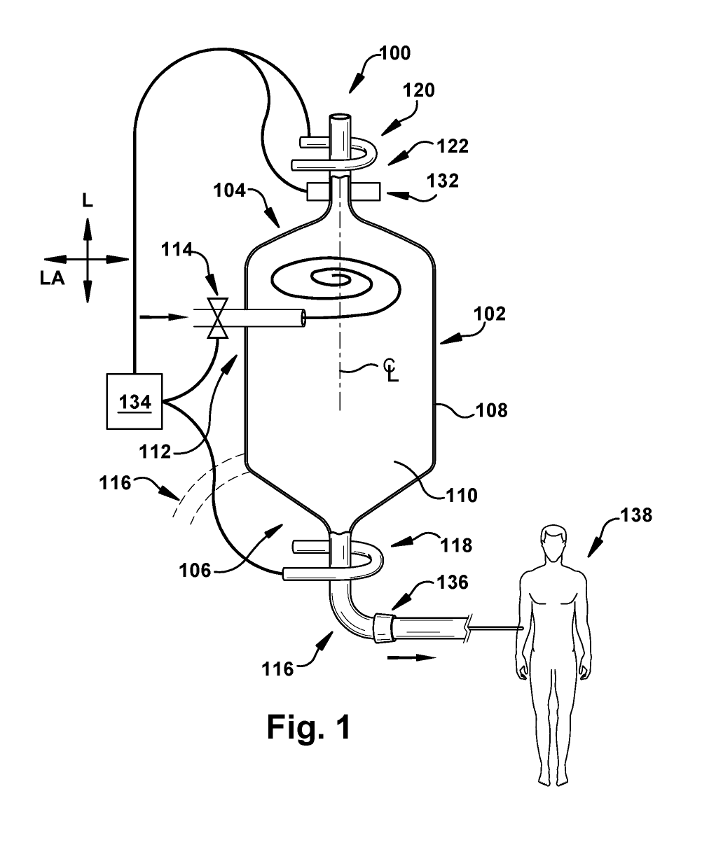

FIGS. 1-3 depict various configurations of an apparatus 100 for removing air from a fluid. The apparatus 100 includes a vertically oriented chamber 102 having longitudinally spaced top and bottom chamber portions 104 and 106, respectively, separated by a chamber sidewall 108. The chamber sidewall 108 at least partially defines a chamber interior 110 having a longitudinally oriented chamber centerline CL. As used herein, a “longitudinal” direction is substantially parallel to arrow “L”, and is the vertical direction in FIGS. 1-3.

The chamber interior 110 could have any suitable dimensions. For example, the chamber interior 110 could have a height in the range of between about 90-110 mm, and more specifically 100 mm. The chamber sidewall 108 will, for most use environments of the apparatus 100, have a circular cross-section with any desired diameter. For example, the chamber sidewall 108 could be in the range of between about 15-25 mm such as, but not limited to, 20 mm. It is contemplated that the circular cross-section could vary in size along the chamber centerline CL; for example, the chamber interior 110 could have a diameter of about 20 mm at the top chamber portion 104 and taper to a diameter of about 18 mm at the bottom chamber portion 106.

A fluid inlet 112 extends through the chamber sidewall 108 and is configured to selectively permit ingress of fluid to the chamber interior 110. The fluid inlet 112 is oriented at a tangent to the chamber sidewall 108, such that fluid entering the chamber interior 110 through the fluid inlet 112 is directed laterally between the chamber sidewall 108 and the chamber centerline CL. A selectively operable fluid inlet valve 114 may selectively permit fluid ingress to the chamber interior 110 via the fluid inlet 112. The fluid inlet 112 may be oriented substantially in a lateral direction relative to the chamber sidewall 108.

It is contemplated that a plurality of fluid inlets 112 could be provided to facilitate entry of the fluid from multiple positions tangent to the chamber interior 110. When present, the plurality of fluid inlets 112 may help to enhance vortex swirling and balance injection forces exerted on the apparatus 100, which may help to prevent unwanted motion or loss of orientation of the apparatus when hanging from a fluid bag or another suspended use environment.

A fluid outlet 116 is located proximate the bottom chamber portion 106 and is configured to selectively permit egress of fluid from the chamber interior 110. A selectively operable fluid outlet valve 118 may selectively permit fluid passage via the fluid outlet 116 to the patient receiving the fluid. The fluid outlet 116 may be oriented substantially in a longitudinal direction relative to the bottom chamber portion 106.

As shown in FIG. 1, the fluid outlet 116 may be located in the bottom chamber portion 106—that is, in a bottommost position in the chamber 102. FIG. 1 also shows, in dashed line, an example of a fluid outlet 116′ which is located in the chamber sidewall 108 longitudinally spaced from the bottom chamber portion 106.

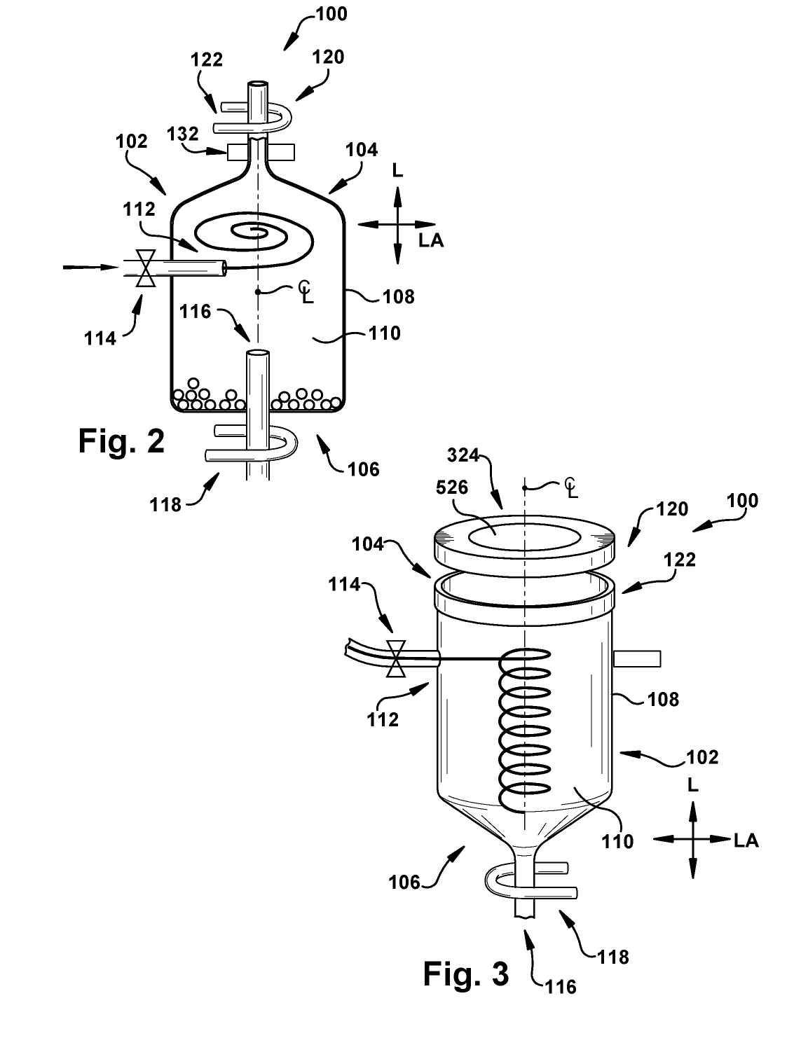

FIG. 2 depicts a situation in which fluid outlet 116 is suspended in the chamber interior 110 longitudinally spaced from the bottom chamber portion 106 and may be, as shown, laterally spaced from the chamber sidewall 108. (The “lateral” direction, as used herein, is substantially perpendicular to the longitudinal direction and is represented by arrow “LA” in the Figures.) The arrangement of FIG. 2 may be useful, for example, when some particulate matter (e.g., clots) is allowed to “settle out” and accumulate on the bottom chamber portion 106 but is undesirable to include in the fluid outlet 116 flow.

An air release structure 120 is located proximate the top chamber portion 104 and is configured to selectively release accumulated air from the chamber interior 110. A selectively operable air release valve 122 may selectively permit release of accumulated air from the chamber interior 110 into ambient space via the air release structure 120.

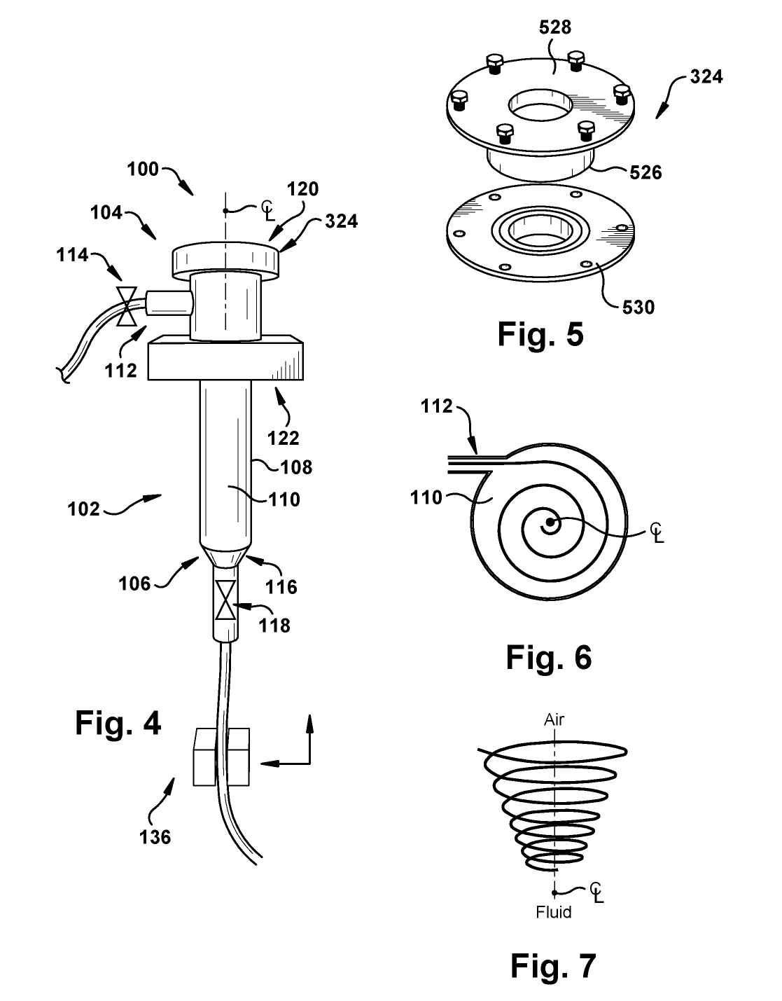

FIGS. 3-4 depict similar aspects of the present invention. In the apparatuses 100 in both FIGS. 3 and 4, an air filter 324 is interposed in a fluid path between at least a portion of the chamber interior 110 and the ambient space, and the air filter 324—when present—serves as at least a portion of the air release structure 120. FIG. 5 is an exploded view of an example configuration of the air filter 324. A filter membrane 526 is sandwiched, or interposed, longitudinally between a top ring 528 and a bottom ring 530, which hold the flexible filter membrane 526 in place. The filter membrane 526 may be made of any desired material, though for many use environments of the apparatus 100, the filter membrane 526 will be made of a hydrophobic material with a pore size between about 0.5-1.5 μm and, more specifically, about 1.0 μm. However, the filter membrane 526 may readily be chosen by one of ordinary skill in the art to semi-permeably allow air (or another gas) to pass therethrough while remaining substantially impervious to the fluid in the apparatus 100. It is contemplated that the filter membrane 526 may also help to prevent entry of contaminants to the fluid in the apparatus 100 from ambient space, as compared to at least an open-topped chamber interior 110 configuration option.

In the embodiment of FIGS. 3-4, a level of fluid within the chamber interior 110 may remain substantially longitudinally coincident with a position of the air filter 324 within the chamber interior 110. Air will therefore be able to escape the chamber interior 110 by passing through the filter membrane 526, while the air filter 324 does not permit fluid passage therethough. In the aspects of the present invention as configured in FIGS. 3-4, air can therefore egress the chamber interior 110 relatively constantly during operation of the apparatus 100 due to the presence of the air filter, without interruption to the flow of fluid through the apparatus 100.

In contrast, in the apparatuses 100 as configured in FIGS. 1-2, the air release valve 122 is selectively operated to facilitate release of accumulated air from the chamber interior 110 into ambient space via the air release structure 120. The flow of fluid through the apparatus 100 is briefly stopped (e.g., via actuation of the fluid outlet valve 118) during release of the accumulated air responsive to operation of the air release valve 122, so that ongoing fluid flow does not cause air to be drawn back down into the chamber interior 110 from ambient space while the air release valve 122 is open. Thus, the fluid flow may be interrupted for a very short period of time for air “venting” during operation of the apparatuses 100 as shown in FIGS. 1-2.

In at least the apparatuses 100 of FIGS. 1-2, an air sensor 132 may be provided for detecting accumulated air in the chamber interior 110. Once the air sensor 132 detects a predetermined amount of air in the chamber interior 110, a user can be alerted so that the air release valve 122 can be opened. Alternatively or additionally, a control system (shown schematically at 134) can be provided for selectively operating the air release valve 122 responsive to the detected accumulated air (e.g., from the air sensor 132) in the chamber interior 110.

More broadly, at least one of the fluid inlet valve 114, the fluid outlet valve 118, and the air release valve 122 could be an electromagnetically operated valve which cooperatively controls fluid passage through a respective one of the fluid inlet 112, the fluid outlet 116, and the air release structure 120. In such case, the control system 134 can selectively operate the electromechanically operated valve(s). This arrangement is shown schematically in FIG. 1.

As shown schematically in FIG. 1, an air detector 136 may be located in a fluid path between the fluid outlet 116 and a patient 138 to whom the fluid is being supplied. The air detector 136, when present, may be used for research purposes and/or can indicate to a user whether the apparatus is operating properly (i.e., is removing air from the fluid as desired). For example, the air detector 136 could detect bubbles having sizes between about 5-15 μm and, more specifically, about 10 μm.

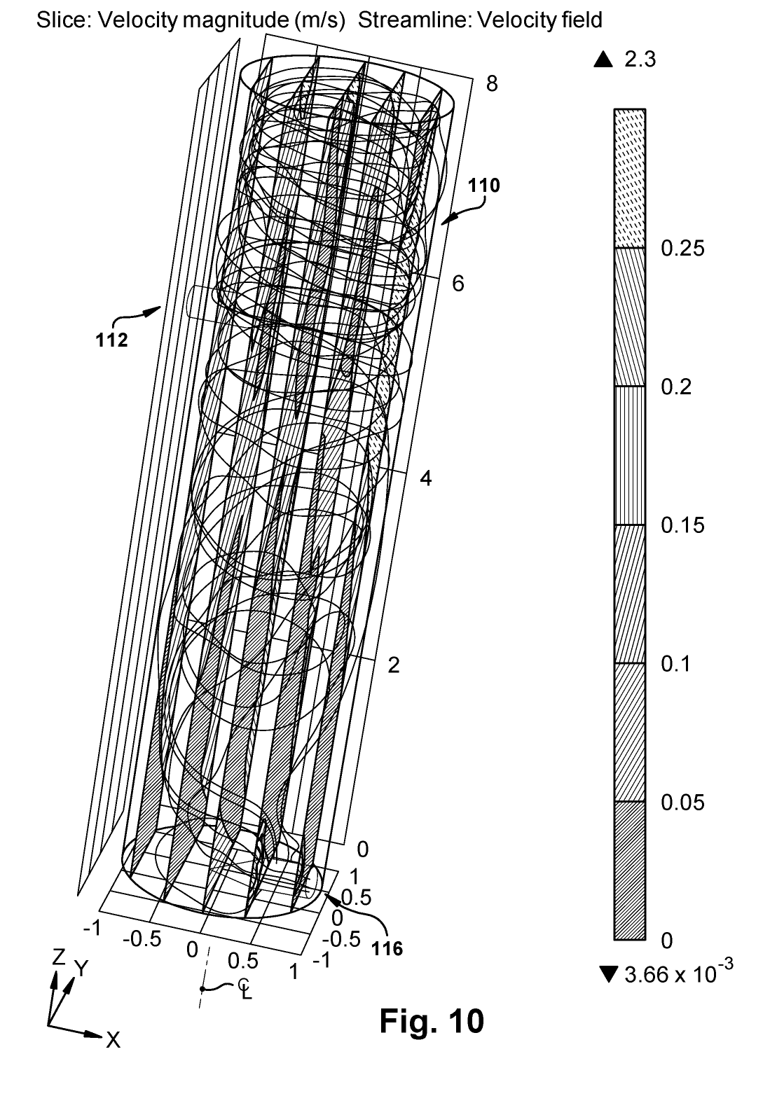

Turning now to FIGS. 6-7, top and side schematic diagrams, respectively, are provided to show the general direction of fluid and air behavior within the chamber interior 110. As shown in FIG. 6, the tangential or off-centerline insertion or ingress of fluid causes a “swirling” or circular motion in the lateral plane. FIG. 7 shows the side view, where gravity urges the swirling fluid downward toward the fluid outlet 116. Accordingly, fluid entering the chamber interior 110 through the fluid inlet 112 achieves a vortex motion during travel downward to the fluid outlet 116. The vortex motion prompts release of air from the fluid during its passage through the chamber interior 110 between the fluid inlet 112 and the fluid outlet 116. (The air could be from any source, and could become present in the fluid at any time, intentionally or not, before the fluid/air mixture enters the chamber interior 110.) The accumulated air collects adjacent the air release structure 120 for selective release. Stated slightly differently, the rotational motion of the fluid in the chamber interior 110 creates different centrifugal forces for the fluid and air because the air has a lower density than the fluid. As a result, the air tends to migrate toward the centerline CL and ascend while the fluid descends in the vortex motion. This dynamic is shown in technical detail in FIG. 10.

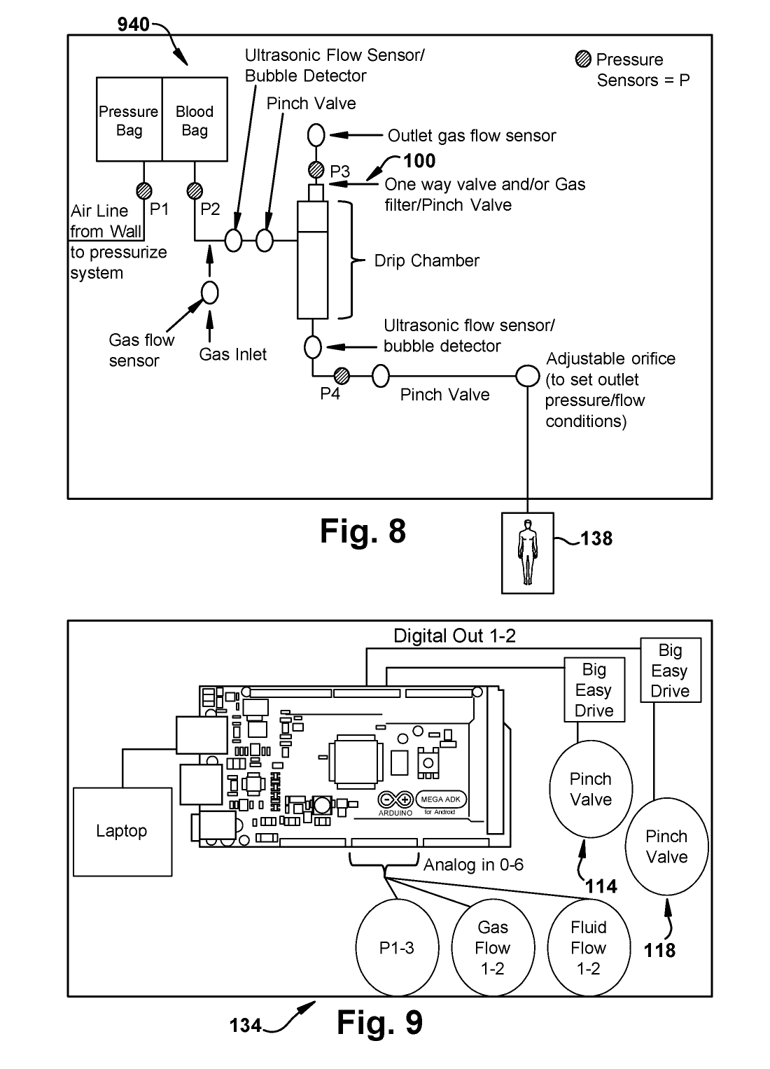

As already alluded to with reference to FIG. 1, the apparatus 100 may be arranged in a fluid path between an intravenous fluid source 940 and a patient 138 to whom the fluid is being supplied. This arrangement is shown in schematic detail in FIG. 8, with various configuration options labeled in that Figure. Likewise, FIG. 9 schematically depicts an example control system 134 which can be used to at least partially automatically control aspects of the operation of the apparatus 100.

A method of removing air from a fluid will now be briefly described, with reference to the above description of the apparatus 100 and the accompanying Figures. First, fluid is selectively permitted ingress to the chamber interior 110 via the fluid inlet 112. The fluid entering the chamber interior 110 is directed through the fluid inlet 112 in a direction laterally between the chamber sidewall 108 and a longitudinally oriented chamber centerline CL. The fluid is then selectively permitted egress of fluid from the chamber interior 110 via the fluid outlet 116.

A vortex motion is imparted to fluid traveling through the chamber interior 110 between the fluid inlet 112 and the fluid outlet 116. With the vortex motion, release of air from the fluid traveling through the chamber interior 110 is prompted to produce accumulated air. The accumulated air is collected in the chamber interior 110 adjacent the air release structure 120. The accumulated air is selectively released from the chamber interior 110 via the air release structure 120.

When the apparatus 100 is of the type shown in FIGS. 3-4, with the air filter 324 interposed in the fluid path between at least a portion of the chamber interior 110 and the ambient space, the accumulated air may dwell within the chamber interior 110 adjacent the air release structure 120 for only a very short amount of time before the accumulated air permeates through the filter membrane 526 and escapes. Thus, the “collection” of accumulated air may be thought of, in the “air filter” arrangement, more as an assemblage of the air released from the fluid at the air filter 324, with substantially continuous release of the accumulated air from the chamber interior 110 via the air filter 324. A level of fluid within the chamber interior 110 may be maintained substantially coincident with a longitudinal position of the air filter 324 within the chamber interior 110, to facilitate removal of air from the apparatus 100 as described herein.

When the apparatus 100 is instead of the type shown in FIGS. 1-2, the presence of a predetermined amount of accumulated air may be sensed in the chamber interior 110. The accumulated air may then be selectively released from the chamber 102 via the air release structure 122, responsive to the sensed presence of the predetermined amount of accumulated air.

As mentioned earlier with reference to FIG. 2, the fluid outlet 116 may be longitudinally spaced within the chamber interior 110 from the bottom chamber portion 106. In this case, the method may include accumulating solid debris (e.g., clots) longitudinally between the fluid outlet 116 and the bottom chamber portion 106, for later removal.

When the apparatus 100 includes at least one electromechanically operated valve (e.g., the fluid inlet valve 114, fluid outlet valve 118, and/or air release valve 122), as mentioned earlier, the method may include cooperatively controlling, with each electromechanically operated valve, fluid passage through a chosen one of the fluid inlet 112, the fluid outlet 116, and the air release structure 120. The at least one electromechanically operated valve may be selectively operated with a control system 134.

The apparatus 100, as shown and described herein in various combinations and aspects, may be used to help remove air from a fluid before the fluid reaches a patient. Accordingly, instances of air embolism in the patient (arising from infused fluid) may be reduced.

While aspects of this disclosure have been particularly shown and described with reference to the example aspects above, it will be understood by those of ordinary skill in the art that various additional aspects may be contemplated. For example, the specific methods described above for using the apparatus are merely illustrative; one of ordinary skill in the art could readily determine any number of tools, sequences of steps, or other means/options for placing the above-described apparatus, or components thereof, into positions substantively similar to those shown and described herein. In an effort to maintain clarity in the Figures, certain ones of duplicative components shown have not been specifically numbered, but one of ordinary skill in the art will realize, based upon the components that were numbered, the element numbers which should be associated with the unnumbered components; no differentiation between similar components is intended or implied solely by the presence or absence of an element number in the Figures. Any of the described structures and components could be integrally formed as a single unitary or monolithic piece or made up of separate sub-components, with either of these formations involving any suitable stock or bespoke components and/or any suitable material or combinations of materials; however, the chosen material(s) should be biocompatible for many applications. Any of the described structures and components could be disposable or reusable as desired for a particular use environment. Any component could be provided with a user-perceptible marking to indicate a material, configuration, at least one dimension, or the like pertaining to that component, the user-perceptible marking potentially aiding a user in selecting one component from an array of similar components for a particular use environment. A “predetermined” status may be determined at any time before the structures being manipulated actually reach that status, the “predetermination” being made as late as immediately before the structure achieves the predetermined status. The term “substantially” is used herein to indicate a quality that is largely, but not necessarily wholly, that which is specified—a “substantial” quality admits of the potential for some relatively minor inclusion of a non-quality item. Though certain components described herein are shown as having specific geometric shapes, all structures of this disclosure may have any suitable shapes, sizes, configurations, relative relationships, cross-sectional areas, or any other physical characteristics as desirable for a particular application. Any structures or features described with reference to one aspect or configuration could be provided, singly or in combination with other structures or features, to any other aspect or configuration, as it would be impractical to describe each of the aspects and configurations discussed herein as having all of the options discussed with respect to all of the other aspects and configurations. A device or method incorporating any of these features should be understood to fall under the scope of this disclosure as determined based upon the claims below and any equivalents thereof.

Other aspects, objects, and advantages can be obtained from a study of the drawings, the disclosure, and the appended claims.

We claim:

1. An apparatus for removing air from a fluid, the apparatus comprising:
a vertically oriented chamber having longitudinally spaced top and bottom chamber portions separated by a chamber sidewall, the chamber sidewall at least partially defining a chamber interior having a longitudinally oriented chamber centerline;
a fluid inlet provided through the chamber sidewall and configured to selectively permit ingress of fluid to the chamber interior, the fluid inlet being oriented at a tangent to the chamber sidewall, such that fluid entering the chamber interior through the fluid inlet is directed laterally between the chamber sidewall and the chamber centerline;
a fluid outlet located proximate the bottom chamber portion and configured to selectively permit egress of fluid from the chamber interior; and
an air release structure located proximate the top chamber portion and configured to selectively release accumulated air from the chamber interior;
wherein fluid entering the chamber interior through the fluid inlet achieves a vortex motion during travel downward to the fluid outlet, the vortex motion prompting release of air from the fluid, the accumulated air collecting adjacent the air release structure for selective release.

2. The apparatus of claim 1, wherein the fluid outlet is located in the bottom chamber portion.

3. The apparatus of claim 1, wherein the fluid outlet is located in the chamber sidewall longitudinally spaced from the bottom chamber portion.

4. The apparatus of claim 1, wherein the fluid outlet is suspended in the chamber interior longitudinally spaced from the bottom chamber portion.

5. The apparatus of claim 1, including an air filter interposed in a fluid path between at least a portion of the chamber interior and the ambient space, and the air filter serves as at least a portion of the air release structure.

6. The apparatus of claim 1, being arranged in a fluid path between an intravenous fluid source and a patient to whom the fluid is being supplied.

7. The apparatus of claim 1, including a selectively operable fluid inlet valve for selectively permitting fluid ingress to the chamber interior via the fluid inlet.

8. The apparatus of claim 1, including a selectively operable fluid outlet valve for selectively permitting fluid passage via the fluid outlet to the patient receiving the fluid.

9. The apparatus of claim 1, including a selectively operable air release valve for selectively permitting release of accumulated air from the chamber interior into ambient space via the air release structure.

10. The apparatus of claim 1, including at least one electromechanically operated valve, each electromechanically operated valve cooperatively controlling fluid passage through a chosen one of the fluid inlet, the fluid outlet, and the air release structure.

11. The apparatus of claim 10, including a control system for selectively operating the at least one electromechanically operated valve.

12. The apparatus of claim 1, including an air detector located in a fluid path between the fluid outlet and a patient to whom the fluid is being supplied.

13. The apparatus of claim 1, including an air sensor for detecting accumulated air in the chamber interior.

14. The apparatus of claim 13, including a selectively operable air release valve for selectively permitting release of accumulated air from the chamber interior into ambient space via the air release structure and a control system for selectively operating the air release valve responsive to the detected accumulated air in the chamber interior.

15. A method of removing air from a fluid, the method comprising:
providing an apparatus including
a vertically oriented chamber with a chamber interior at least partially defined by longitudinally spaced top and bottom chamber portions separated by a chamber sidewall, the chamber sidewall at least partially defining the chamber interior,
a fluid inlet provided through the chamber sidewall, the fluid inlet being oriented at a tangent to the chamber sidewall,
a fluid outlet located proximate the bottom chamber portion, and
an air release structure located proximate the top chamber portion;
selectively permitting ingress of fluid to the chamber interior via the fluid inlet;
directing fluid entering the chamber interior through the fluid inlet in a direction laterally between the chamber sidewall and a longitudinally oriented chamber centerline;
selectively permitting egress of fluid from the chamber interior via the fluid outlet;
imparting a vortex motion to fluid traveling through the chamber interior between the fluid inlet and the fluid outlet;
with the vortex motion, prompting release of air from the fluid traveling through the chamber interior to produce accumulated air;
collecting the accumulated air in the chamber interior adjacent the air release structure; and
selectively releasing the accumulated air from the chamber interior via the air release structure.

16. The method of claim 15, wherein the fluid outlet is longitudinally spaced within the chamber interior from the bottom chamber portion, the method including accumulating solid debris longitudinally between the fluid outlet and the bottom chamber portion.

17. The method of claim 15, including
sensing the presence of a predetermined amount of accumulated air in the chamber interior; and
selectively releasing the accumulated air from the chamber via the air release structure responsive to the sensed presence of the predetermined amount of accumulated air.

18. The method of claim 15, including an air filter interposed in a fluid path between at least a portion of the chamber interior and the ambient space, and the air filter serves as at least a portion of the air release structure, and wherein the method includes maintaining a level of fluid within the chamber interior substantially coincident with a position of the air filter within the chamber interior.

19. The method of claim 15, wherein the apparatus includes at least one electromechanically operated valve, the method including cooperatively controlling, with each electromechanically operated valve, fluid passage through a chosen one of the fluid inlet, the fluid outlet, and the air release structure.

20. The method of claim 19, including selectively operating the at least one electromechanically operated valve with a control system.
